(19) United States
(12) Patent Application Publication
(10) Pub. No.: US 2024/0335670 A1
(43) Pub. Date: Oct. 10, 2024

(54) IMPLANTABLE ANTENNA AND COEXISTING SENSOR CONFIGURATIONS

(71) Applicant: Oracle Health, Inc., Safety Harbor, FL (US)

(72) Inventors: David Nghiem, Shoreview, MN (US); Jaeson Bang, Safety Harbor, FL (US); J. Christopher Flaherty, Nottingham, NH (US)

(21) Appl. No.: 18/579,954

(22) PCT Filed: Jul. 28, 2022
(86) PCT No.: PCT/US2022/038653

§ 371 Date: Jan. 17, 2024

Related U.S. Application Data
(60) Provisional application No. 63/259,912, filed on Jul. 28, 2021.
(60) Provisional application No. 63/321,936, filed on Mar. 21, 2022.

(51) Int. Cl. A61N 1/372

(52) U.S. Cl. CPC A61N 1/37229

(57) ABSTRACT

Various antenna configurations are disclosed. For example, a conductive structure included as a portion of an implantable antenna can include a first portion extending along a first plane, and a second portion extending along a second plane, the second plane orthogonal to the first plane. In addition to the antenna configurations described herein, various configurations are disclosed for electrode configurations, such as can be arranged to avoid interference or parasitic loading of the antenna structures described herein, in an implantable device application. Such electrode configurations can be used for physiologic sensing, such as for sending an electrocardiogram or other physiologic signal. For example, an implantable device can include a sensor electrode structure defined by at least two conductive regions that are conductively isolated and capacitively coupled with each other.

### RELATED APPLICATIONS

This application claims benefit to U.S. Provisional Application Ser. No. 63/259,912 (Docket No.: OCL-003-PR1), titled “IMPLANTABLE ANTENNA AND SENSOR CONFIGURATIONS”, filed Jul. 28, 2021, the content of which is incorporated by reference in its entirety.

This application claims benefit to U.S. patent application Ser. No. 17/443,899 (Docket No.: OCL-003-US), titled “IMPLANTABLE ANTENNA AND SENSOR CONFIGURATIONS”, filed Jul. 28, 2021, the content of which is incorporated by reference in its entirety.

This application claims benefit to U.S. Provisional Application Ser. No. 63/321,936 (Docket No.: OCL-002-PR1), titled “IMPLANTABLE CARDIAC MONITOR”, filed Mar. 21, 2022, the content of which is incorporated by reference in its entirety.

This application is related to U.S. Provisional Application Ser. No. 62/853,899 (Docket No.: OCL-001-PR1), titled “IMPLANTABLE CARDIAC MONITOR”, filed May 29, 2019, the content of which is incorporated by reference in its entirety.

This application is related to U.S. patent application Ser. No. 17/611,335 (Docket No.: OCL-001-US), titled “IMPLANTABLE CARDIAC MONITOR”, filed Nov. 15, 2021, United States Publication Number 2022-0192600, published Jun. 23, 2022, the content of which is incorporated by reference in its entirety.

This application is related to International PCT Patent Application Serial Number PCT/US2020/035171 (Docket No.: OCL-001-PCT), titled “IMPLANTABLE CARDIAC MONITOR”, filed May 29, 2020, United States Publication Number WO2020/243463, published Dec. 3, 2020, the content of which is incorporated by reference in its entirety.

### FIELD OF THE INVENTIVE CONCEPTS

This document pertains generally, but not by way of limitation, to antenna configurations, and more particularly to antennas that can be used in implantable devices, such as coexisting with a sensor.

### BACKGROUND

Implantable medical devices can include circuitry to sense one or more physiologic signals. In addition or instead, such implantable medical devices can include circuitry to provide electrostimulation or trigger drug therapy, as illustrative examples. Monitoring features of an implantable medical device can include detection of physiologic events or logging of received physiologic signals. In one approach, retrieval of stored representations of such signals or real-time transmission of monitored signals can be accomplished using a near-field magnetic telemetry scheme, such as providing one-way or bi-directional communication between an implantable device and a nearby coupled pickup device (e.g. a wand located externally to the body, at most a few centimeters away from the implantable device). Such a near-field scheme can be used for configuration or control of the implantable device.

### SUMMARY

The present inventors have recognized, among other things, that a near-field communication scheme for transfer of information from or to an implantable device can prove cumbersome for use by a caregiver or patient. A caregiver or patient may be requested to place a wand or other pickup at a specified location nearby an implantable device or communication cannot occur. Use of a wand may entirely preclude retrieval of data from an implantable device in a passive manner without requiring intervention by a patient or caregiver. Instead, when a wand or other user interaction is required, inconvenience or extra costs may be incurred at follow-up, or locations may be limited in terms of where online/remote follow up can be performed. Accordingly, the present inventors have developed, among other things, an antenna configuration suitable for use in an implantable device to facilitate longer-range (radiative) communication with other devices. For example, such an implantable antenna configuration can be sized and shaped to facilitate use of an operating frequency range allocated within an Industrial, Scientific, and Medical (ISM) band, as an illustrative example. In an illustrative example, an implantable medical device (e.g. an active or other implantable medical device) such as an implantable monitoring device, can include a transceiver certified for compatibility with a Bluetooth® standard, such as conforming to a Bluetooth® Low Energy (BLE) specification.

Use of the antenna configurations described herein can facilitate longer-range (e.g. meters or even tens of meters) communication without requiring a near-field “repeater” or other intermediary device external to the patient. Moreover, compatibility of antenna configurations described herein with standard communication schemes such as BLE facilitates potential interoperability with a broad range of BLE-enabled devices, such as cellular devices, tablets, mobile devices, portable or desktop computers, or application-specific monitoring devices such as BLE-enabled bed-side monitors.

In addition to the antenna configurations described herein, various configurations are disclosed for electrode configurations, such as can be arranged to avoid interference or parasitic loading of the antenna structures described herein, in an implantable device application. Such electrode configurations can be used for physiologic sensing, such as for sending an electrocardiogram or other physiologic signal.

In an example, an implantable antenna assembly comprises a feed location, a dielectric portion, and a conductive structure located on or within the dielectric portion, the conductive structure coupled to the feed location. In an example, the conductive structure includes a first portion extending along a first plane, and a second portion extending along a second plane, the second plane orthogonal to the first plane. In an example, at least one of the first or second portions projects in both a parallel and a perpendicular in-plane direction, with respect to an in-plane reference axis. In an example, the first portion comprises about one third of a total length including the first and second portions, and the second portion comprises about two thirds of the total length including the first and second portions.

In an example, the implantable antenna assembly optionally includes at least one of the first portion or the second portion being divided into segments extending in different directions. For example, the first portion is optionally divided into a first segment and a second segment, where a length of the first segment is about one third of a length of the first portion, and a length of the second segment is about two thirds of the length of the first portion.

In an example, an implantable device includes an antenna assembly, the implantable device comprising a conductive housing, defining a feed location, a dielectric portion mechanically coupled to the conductive housing, an antenna structure defined by a conductive structure located on or within the dielectric portion, the conductive structure comprising a first portion extending along a first plane and a second portion extending along a second plane, the second plane orthogonal to the first plane, and where at least one of the first or second portions projects in both a parallel and a perpendicular in-plane direction, with respect to an in-plane reference axis. In an example, the implantable device includes a sensor electrode structure defined by at least two conductive regions that are conductively isolated and capacitively coupled with each other.

In some embodiments, the implantable device comprises a shielding assembly. The shielding assembly can comprise one, two, or more thermal shielding components and/or one, two, or more electromagnetic shielding elements. The shielding assembly can comprise shield material that is positioned on a side of the implantable device that is facing away from the patient's skin. The shielding assembly can comprise one, two, or more electromagnetic shielding components comprising radio-absorptive shield material and/or radio-reflective shield material.

In some embodiments, the implantable device further comprises a functional element comprising one, two, or more functional elements. The functional element can comprise one, two, or more sensors and/or one, two or more transducers. The functional element can be configured to provide a therapeutic function. The functional element can be configured to deliver therapeutic energy and/or a therapeutic agent. The functional element can be configured to reduce MRI effects. The functional element can comprise one, two, or more components selected from the group consisting of: heat sink; heat spreader; shielding; high heat conduction element; active shorting element; passive shorting element; reed switch; mechanical switch; switch activated before and/or during MRI use; parallel electrical connections; current diverter; and combinations thereof.

In some embodiments, the implantable device further comprises an algorithm assembly, the algorithm assembly comprising a controller and a memory storage component coupled to the controller, and the memory storage component stores instructions for the controller to perform an algorithm. The algorithm can comprise one, two, or more artificial intelligence algorithms. The algorithm can comprise one, two, or more algorithms configured to adjust one or more antenna parameters of the implantable device. The algorithm can comprise one, two, or more algorithms configured to perform a diagnosis and/or deliver a therapy.

In some embodiments, the implantable device comprises one or more sensors configured to record physiologic data and/or other data, and the algorithm is configured to perform the diagnosis and/or deliver the therapy in a closed loop arrangement based on the recorded physiologic data and/or other data.

This summary is intended to provide an overview of subject matter of the present patent application. It is not intended to provide an exclusive or exhaustive explanation of the invention. The detailed description is included to provide further information about the present patent application.

### INCORPORATION BY REFERENCE

All publications, patents, and patent applications mentioned in this specification are herein incorporated by reference to the same extent as if each individual publication, patent, or patent application was specifically and individually indicated to be incorporated by reference. The content of all publications, patents, and patent applications mentioned in this specification are herein incorporated by reference in their entirety for all purposes.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates generally a system that can include an implantable medical device comprising an implantable antenna assembly.

FIG. 2A, FIG. 2B, FIG. 2C, and FIG. 2D illustrate respective examples comprising implantable antenna assembly configurations, such as can be included in an implantable medical device or form a portion of an implantable medical device.

FIG. 3A and FIG. 3B illustrate further respective examples comprising implantable antenna assembly configurations, such as can be included in an implantable medical device or form a portion of an implantable medical device.

FIG. 4 illustrates generally an illustrative example of a distal end of a conductive structure that can form a portion of an implantable antenna assembly.

FIG. 5 illustrates generally an example comprising a sensor electrode structure.

FIG. 6A shows an illustrative example of a simulated surface current density for a dipole antenna configuration at an operating frequency of 2.45 GHz.

FIG. 6B shows an illustrative example of a simulated surface current density for a dipole antenna configuration at an operating frequency of 2.45 GHz and an induced surface current density for a nearby unsegmented sensor electrode structure.

FIG. 6C shows an illustrative example of a simulated surface current density for a dipole antenna configuration at an operating frequency of 2.45 GHz and an induced surface current density for a nearby segmented sensor electrode structure.

FIG. 6D shows an illustrative example of a simulated surface current density for a dipole antenna configuration at an operating frequency of 2.45 GHz and an induced surface current density for a nearby unsegmented sensor electrode structure, where the sensor electrode structure is oriented in a direction perpendicular to a long axis of the implantable antenna structure.

FIG. 7A illustrates generally an example comprising a sensor electrode structure having two layers of conductive regions.

FIG. 7B shows an illustrative example of a simulated surface current density for a dipole antenna configuration at an operating frequency of 2.45 GHz and an induced surface current density for a nearby two-layer segmented sensor electrode structure.

FIG. 8A, FIG. 8B, and FIG. 8C show respective illustrative examples of arrays of sensor electrode structures having conductive regions extending in two directions (e.g. a two-dimensional array structure).

### DETAILED DESCRIPTION

Various challenges can be presented in relation to use of radiating communication antenna configurations on and/or within implantable medical devices (e.g. active implantable medical devices). For example, a relative dielectric constant (e.g. relative permittivity) of muscle tissue, or a combination of muscle and fat tissue, is generally much greater than unity at frequencies normally used for wireless communications (e.g. from a range of tens of megahertz (MHz) to about 5 gigahertz (GHz)). Muscle tissue is also generally lossy and dispersive (e.g. such a medium can present a much higher conductivity than free space and the dielectric properties of such material vary substantially with respect to frequency). A tissue medium may also exhibit different propagation characteristics depending on a dominant polarization of an implantable antenna configuration. Accordingly, the present inventors have recognized, among other things, that an implantable antenna configuration can be arranged to provide polarization diversity while still providing a compact antenna configuration. Such a compact configuration is facilitated by a shorter “effective wavelength” of electromagnetic waves within the specified operating frequency range, taking into account a higher relative dielectric constant of the surrounding tissue medium.

Generally, the phrase “effective wavelength” refers to a wavelength as seen by the antenna when surrounded by an inhomogeneous medium, such as layers comprising free space, muscle and/or fat tissue, and a dielectric housing. The effective wavelength is generally an intermediate value reflecting an effective relative dielectric constant that is between a relative dielectric constant of a housing and that of the tissue medium (and generally greater in magnitude than the relative dielectric constant of the housing).

Generally, a length of an antenna structure (e.g. an effective length as determined either along a conductive structure of the antenna and/or defined by a diameter of a sphere bounding the largest dimension of the antenna structure) can be specified based at least in part upon an intended operational frequency range. For a monopole antenna structure or monopole-like structure, an overall length of the antenna structure can be specified to be a quarter of an effective wavelength, or an odd multiple thereof. For a dipole antenna structure, or a dipole-like structure, an overall length of the antenna structure can be specified to be half of an effective wavelength.

A tradeoff can exist between antenna length, efficiency, and proximity of the antenna to the tissue interface. For example, if an antenna is embedded further within a dielectric housing, the effective wavelength can be longer (and hence the antenna structure is physically larger to achieve a quarter or half wavelength criterion), because the effective relative dielectric constant sees a greater contribution from the dielectric housing relative to a contribution from the tissue medium. Conversely, if the antenna is located in relatively closer proximity to tissue, the effective wavelength is shorter, and the antenna can be made physically smaller. Generally, the antenna configurations disclosed herein can be used in a variety of configurations such as at different depths within a dielectric housing, where the antenna elements can be increased and/or decreased in physical length in accordance with a determined effective wavelength (either via simulation, or empirically, or both).

As an illustration, a dipole antenna can be embedded within a 6 millimeter (mm)-thick dielectric compartment having a relative dielectric constant of about 4, such as a compartment that is implanted 15 mm below tissue (e.g. in a torso location) along a first face of the dielectric compartment, with 200 mm of tissue depth below a second face of the dielectric compartment (e.g. through the remainder of the torso to the back). If the relative dielectric constant is assumed to be about 53, and the tissue conductivity is assumed to be about 1.8 Siemens per meter, antenna efficiencies and lengths can be simulated. For example, if the antenna is embedded 1 mm within the dielectric compartment facing the first surface, an antenna length for optimal match at 2.45 GHz is about 26 mm, with an efficiency of about 0.42%. Similarly, if the antenna is located just below or at the surface of the dielectric compartment, an antenna length for optimal match at 2.45 GHz is about 14.5 mm, with an efficiency of about 0.20%. Such examples illustrate that as an antenna is located closer to the tissue medium, the effective dielectric constant increases, resulting in a shorter effective wavelength, a shorter physical antenna length for optimal matching, but reduced radiative efficiency.

The configurations of the present inventive concepts shown herein can provide improved performance even when located at or near a surface of a dielectric compartment, such as in part by providing polarization diversity by using segments and/or portions providing radiation having orthogonal polarization in three orthogonal axes. Radiation in different polarization axes facilitates reception because it may help to reduce cross-polarization losses, such as where a transmitter and/or receiver is not matched to a polarization mode of a counterpart receiver and/or transmitter.

Lengths of respective antenna portions can be established such as by considering a current distribution during operation. For a simulated and/or empirically determined radiation pattern, antenna portions can be sized to achieve a more uniform (e.g. isotropic) radiation profile, or to achieve an even distribution of radiated power across different polarization axes, or both. Various examples herein show a monopole configuration, but the structures and techniques of the present inventive concepts described herein can be implemented to provide a dipole antenna configuration, such as by providing two arms having similar or symmetrical geometries, such as a first arm defined by a first conductive structure and fed using a first conductor from an antenna port, and a second arm defined by a second conductive structure and fed by a second conductor of the antenna port, such as where the conductive structures are fed using a balanced port configuration.

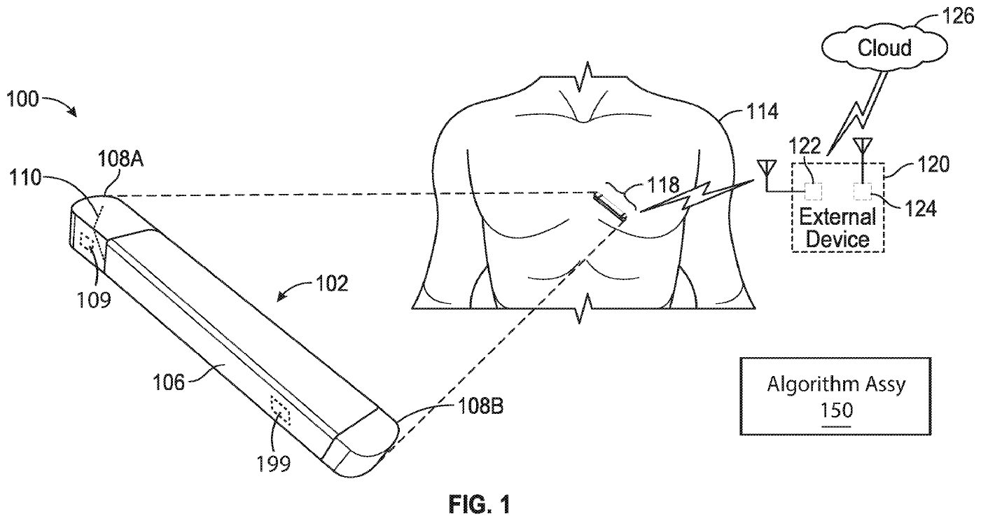

FIG. 1 illustrates generally a system 100 that can include one or more implantable medical devices, implant 102 (e.g. a single device shown). Each implant 102 can comprise an implantable antenna assembly, antenna 110 as shown. The implantable antenna assembly can include a monopole configuration such as embedded in a dielectric portion 108A (e.g. a dielectric compartment or header). A counterpoise can be provided by a separate conductive structure or even by a housing 106 of the implant 102. The dielectric portion 108A can be mechanically coupled to the housing 106, such as using an adhesive or other technique, such as via welding or use of a fastener, or a combination of various techniques. In an example, the antenna 110 can include two separate arms to provide a dipole configuration. In yet another example, a second dielectric portion 108B can be located at an end of the implant 102 opposite the dielectric portion 108A. The second dielectric portion 108B can house an antenna separate from antenna 110 in the dielectric portion 108A or form another arm or element thereof.

In an illustrative example, implant 102 can include an implantable monitoring device, such as implantable in a subcutaneous pectoral location, implant location 118, within a subject 114 (e.g. a person as shown in FIG. 1 as an illustration, but such a monitoring device can also be implanted in an animal such as for agricultural or veterinary use, as illustrative examples). The implant 102 can include one or more physiologic sensors, such as having sensor electrodes located on or within one of the dielectric portion 108A or second dielectric portion 108B, or using one or more conductive portions of the housing 106.

The system 100 can include or can be communicatively coupled with one or more external devices, such as an external device 120 (e.g. a bedside monitor, a mobile device, a tablet, a portable or desktop computer, or the like). The external device can include a first transceiver 122 for communication with implant 102. As mentioned above, the first transceiver 122 can conform to a Bluetooth® specification or other standard, such as to facilitate communication using a BLE protocol at or around 2.45 GHz. The external device 120 can use the first transceiver 122 or another transceiver such as a second transceiver 124 to communicate with other devices, such as a cloud-based repository or other remote repository, cloud 126 shown. For example, the second transceiver 124 can include a wireless networking transceiver or cellular modem, as illustrative examples. Generally, retrieved physiologic data or operating data relating to the implant 102 can be stored, such as for retrieval, review, reporting, or alerting, as illustrative examples.

In some embodiments, implant 102 (e.g. antenna 110) comprises an assembly, shielding assembly 109 shown, that can be configured to shield (e.g. thermally or electromagnetically shield) one or more portions of implant 102. Shielding assembly 109 can comprise one, two, three, or more shielding components, such as one or more thermal shielding components and/or one or more electromagnetic shielding components. Shielding assembly 109 can comprise shield material (e.g. electromagnetic shield material) that is positioned on a side of implant 102 that is facing away from the patient's skin. The shield material can comprise at least one of: radio-absorptive shield material or radio-reflective shield material.

In some embodiments, system 100 (e.g. implant 102) comprises one, two, or more functional elements, functional element 199 shown. Functional element 199 can comprise one, two, or more sensors, and/or one, two or more transducers. Functional element 199 can comprise an assembly or other component configured to provide a therapeutic function, such as a component configured to deliver therapeutic energy (e.g. an electrode, ultrasound delivery component, thermal delivery component, and/or light delivery component) and/or a therapeutic agent (e.g. a needle or other drug delivery component). In some embodiments, functional element 199 comprises a component configured to reduce MRI effects. The MRI effect-reducing functional element 199 can comprise one, two, or more components selected from the group consisting of: heat sink; heat spreader; shielding; high heat conduction element; active shorting element; passive shorting element; reed switch; mechanical switch; switch activated before and/or during MRI use; parallel electrical connections; current diverter; and combinations thereof.

In some embodiments, system 100 (e.g. implant 102) comprises an assembly, algorithm assembly 150 shown, which can be configured to perform one, two, or more algorithms. Algorithm assembly 150 can comprise one or more electronic elements, electronic assemblies, and/or other electronic components, such as components selected from the group consisting of: controllers (e.g. central processing units and/or other controllers); memory storage components; analog-to-digital converters; rectification circuitry; state machines; microprocessors; microcontrollers; filters and other signal conditioners; sensor interface circuitry; transducer interface circuitry; and combinations thereof. In some embodiments, algorithm assembly 150 comprises a controller and a memory storage component (e.g. coupled to the controller). The memory storage component can include instructions, such as instructions used by algorithm assembly 150 to perform an algorithm (e.g. used by a controller of algorithm assembly 150 to perform one or more algorithms). In some embodiments, algorithm assembly 150 comprises one or more algorithms comprising a machine learning, neural net, and/or other artificial intelligence algorithm (“AI algorithm” herein). In some embodiments, algorithm assembly 150 comprises an algorithm (e.g. an AI algorithm) configured to adjust one or more parameters (e.g. drive parameters and/or receive parameters) of antenna 110 (e.g. to transmit and/or receive data in a closed-loop arrangement). In some embodiments, algorithm assembly 150 comprises an algorithm (e.g. an AI algorithm) configured to cause implant 102 to perform a diagnosis, and/or deliver a therapy, in a closed loop arrangement (e.g. based on an analysis of physiologic and/or other sensor signals recorded by implant 102 and/or another component of system 100).

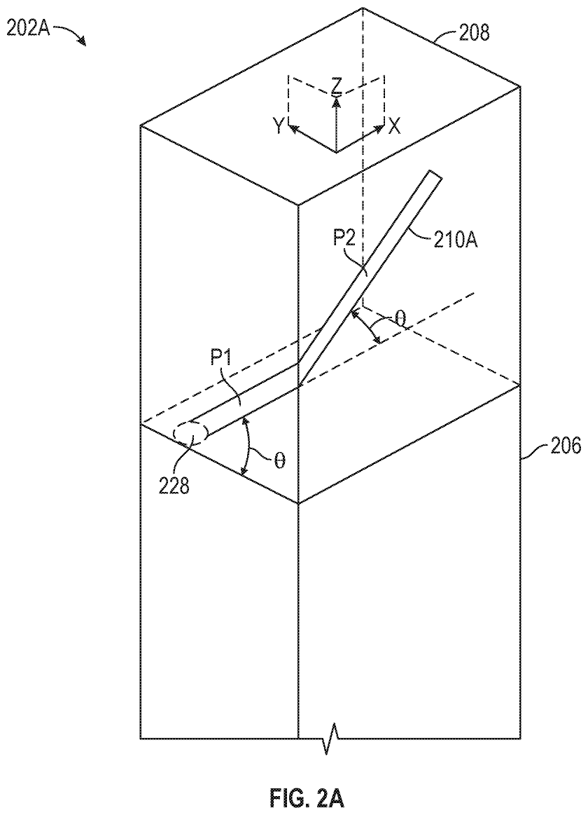

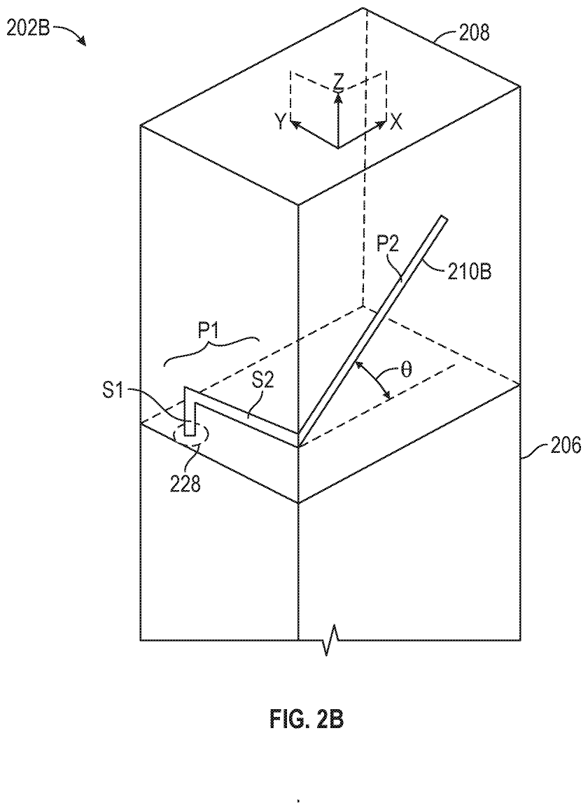

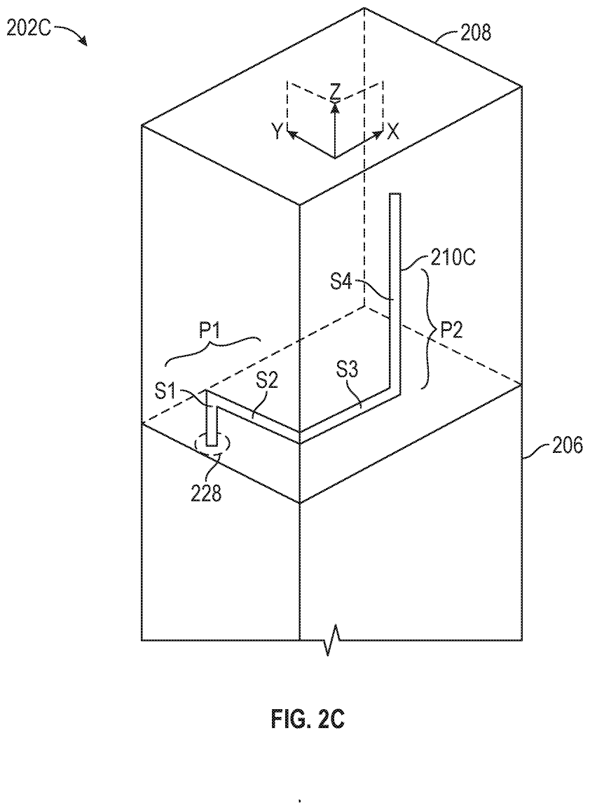

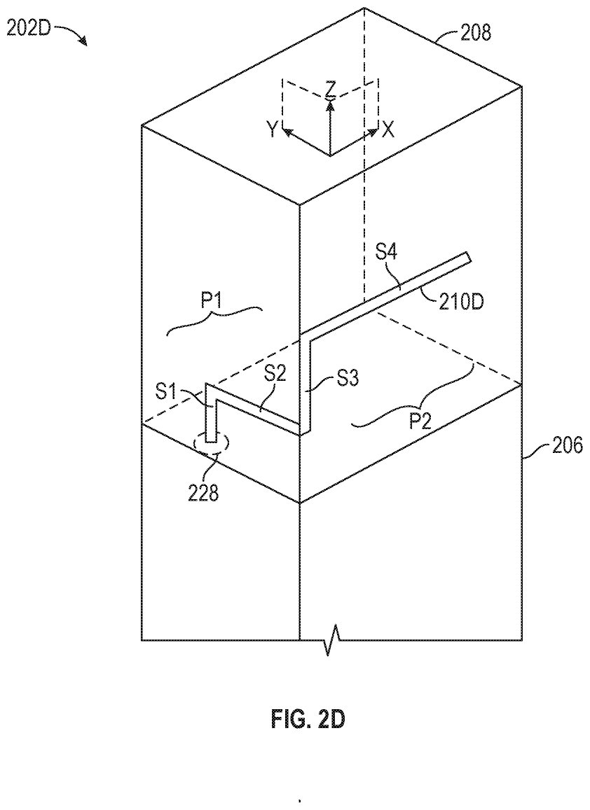

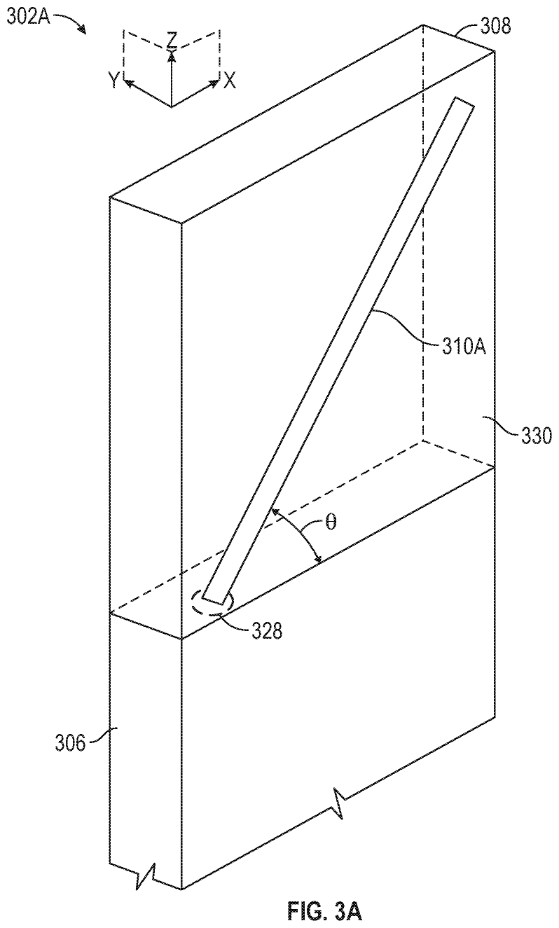

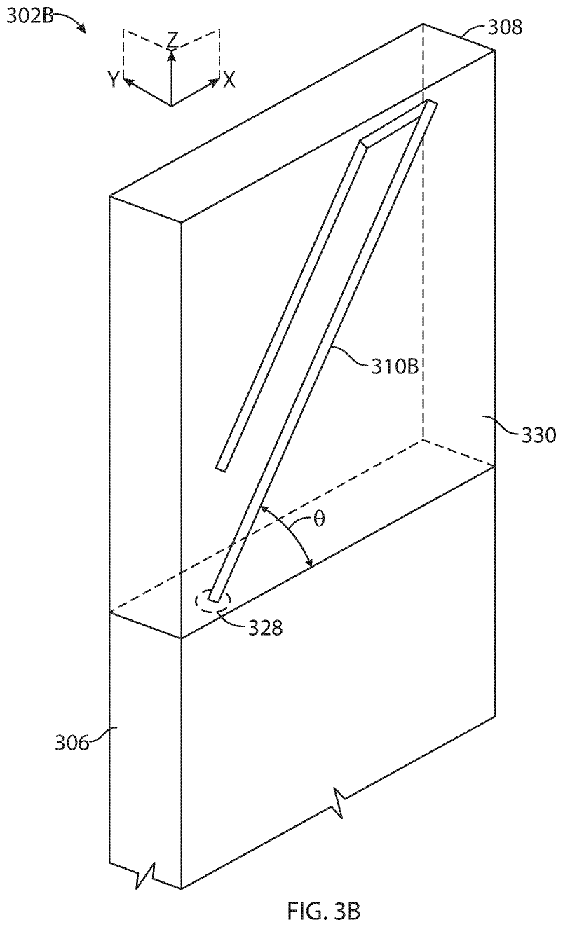

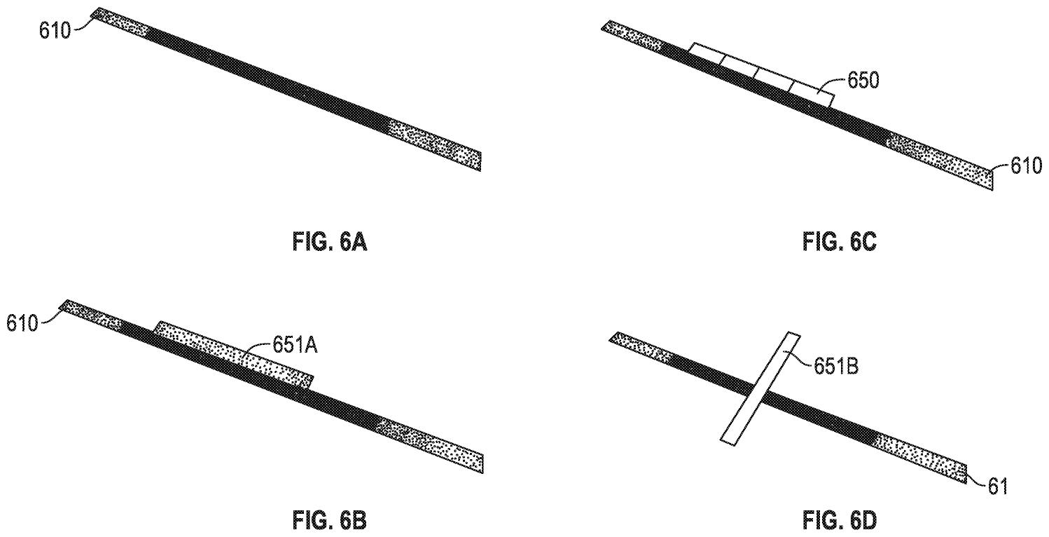

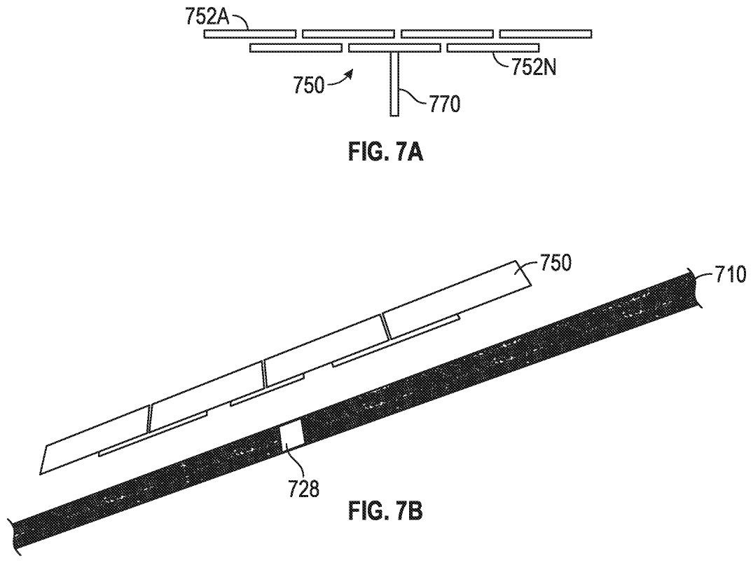

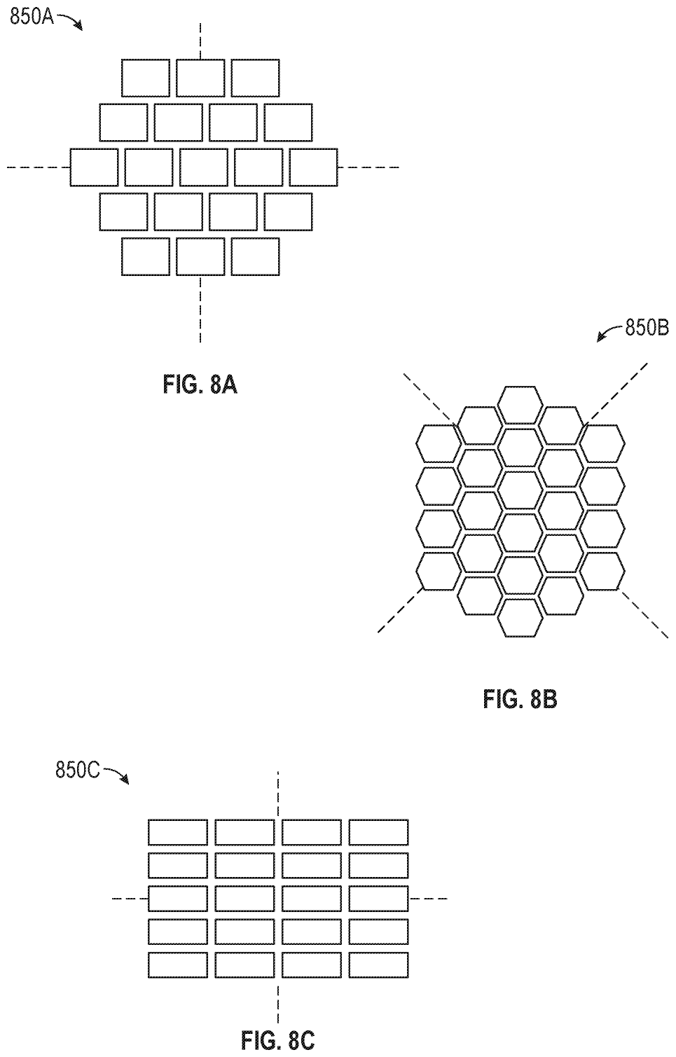

Antenna 110 can include aspects as shown in other examples herein, such as shown and discussed below in FIG. 2A, FIG. 2B, FIG. 2C, FIG. 2D, FIG. 3A, or FIG. 3B, or elsewhere. Electrode configurations that can co-exist with such antenna configurations (among others) are shown illustratively in the examples of FIG. 5, FIG. 6C, FIG. 6D, FIG. 7A, and FIG. 7B. Further variations of such electrode configurations are possible, such as shown illustratively in FIG. 8A, FIG. 8B, and FIG. 8C. Antenna 110 can comprise configurations of one, two, or more electrodes that are positioned in a one-dimensional (e.g. linear), two-dimensional, and/or three-dimensional arrangement.

FIG. 2A, FIG. 2B, FIG. 2C, and FIG. 2D illustrate respective examples comprising implantable antenna assembly configurations, such as can be included in an implantable medical device or form a portion of an implantable medical device. In FIG. 2A, an implantable medical device, implant 202A shown, can include a housing 206 (e.g. a “conductive housing” comprising one or more conductive materials and/or conductive portions), and a dielectric portion 208 (e.g. a header or dielectric compartment, such as a portion comprising a biocompatible polymer such as a polyether-based thermoplastic urethane). The dielectric portion can house a conductive structure 210A forming a portion of an implantable antenna assembly. The conductive structure 210A can have a wire-shaped or ribbon-shaped (e.g. rectangular) profile, as illustrative examples. Generally, the conductive structure 210A can have a first portion P1 extending along a first plane (in this case a Z-Y plane as defined in the coordinate system shown in FIG. 2A), and a second portion P2 extending along a second plane (e.g. a Z-X plane), where the first and second portions P1 and P2 project in both a parallel and perpendicular direction in-plane. For example, the first portion P1 projects in both the Z direction and the Y direction, and the second portion P2 projects in both the Z direction and the X direction. If the Z direction is taken as a reference axis, then the projections of each of the first portion P1 and the second portion P2 are parallel to the Z axis, and perpendicular to the Y and X axes, respectively. Alternatively, if the X axis is regarded as a reference axis, then the second portion P2 projects (in a vector sense) in both a direction parallel to the X axis and a direction perpendicular to the X axis. Similarly, if the Y axis is regarded as a reference axis for the first portion P1, the first portion P1 projects (in a vector sense) in both a direction parallel to Y axis and a direction perpendicular to the Y axis. The reference axes may be defined as an intersection of a third plane (e.g. an X-Y plane) with the first plane and the second plane, where the X-Y plane is orthogonal to the other planes.

Such projection facilitates generation of radiation having linear polarization in three orthogonal axes. To control a balance of radiation across different polarizations, and to control radiation spatially, respective angles theta (0) can be specified, such as defining a proportion of horizontal versus. vertically-oriented polarization for the corresponding portion (corresponding to the orientation of the antenna shown in FIG. 2A, for example). Relative proportions of the total length of the conductive structure 210A can be specified for each of the first and second portions, such as following a “⅓” and/or “⅔” rule. For example, a length of the first portion P1 can be ⅓ of a total length of the conductive structure 210A, and a length of the second portion P2 can be ⅔ of the total length of the conductive structure 210A. In this manner, ideally, one half of the radiated power is associated with the first portion P1 proximal to a feed location 228 and another half of the radiated power is associated with the second portion P2 distal to the feed location 228. The feed location 228 can include an insulated feedthrough structure, such as a coaxial feedthrough configuration providing a pathway for a signal to be fed to the conductive structure 210A from a transceiver located within a sealed (e.g. hermetically sealed) compartment within the housing 206. For the monopole configuration of FIG. 2A, the total length of the conductive structure 210A would generally be specified to be a quarter of an effective wavelength accounting for the contributions of different media nearby the conductive structure 210A.

Again, ideally, if θ is specified to be 45 degrees, ¼ of the total radiated power is generated by the vertically-oriented projection of P2, ¼ of the total radiated power is generated by the vertically-oriented projection of P1, ¼ of the total radiated power is generated by the horizontally-oriented projection of P2, and ¼ of the total radiated power is generated by the horizontally-oriented projection of P1. In practice, the angle θ can be specified to compensate for non-ideal polarization diversity or to provide enhanced radiation in one or two of the three orthogonal polarization axes. Other techniques can be used to provide radiation having multiple polarization contributions.

FIG. 2B illustrates a configuration similar to FIG. 2A, showing an implantable medical device, implant 202B, where a conductive structure 210B is located upon or within a dielectric portion 208, such as located at an end of a housing 206. In FIG. 2B, the conductive structure 210B includes a first portion P1 divided into two segments S1 and S2, where S1 extends in a direction perpendicular to the Y axis, and S2 extends in a direction parallel to the Y axis. The segment S1 can be proximal to a feed location 228 and can be a third of the length of the sum of segment lengths, S1+S2, and the segment S2 can be two-thirds of the length of the sum S1+S2. As in the conductive structure 210A of FIG. 2A, in FIG. 2B, a second portion P2 can be two thirds of the total length of all portions of the conductive structure 210B, where P1 is one third of the total length (e.g. S1+S2=P1). The angle θ can be specified to be 45 degrees as in the example of the conductive structure 210A of FIG. 2A.

FIG. 2C illustrates a further configuration similar to FIG. 2A and FIG. 2B where an implantable medical device, implant 202C shown, can include a conductive structure 210C located upon or within a dielectric portion 208, such as located at an end of a housing 206. In FIG. 2C, the conductive structure 210C includes a first portion P1 divided into two segments S1 and S2, where S1 extends in a direction perpendicular to the Y axis, and S2 extends in a direction parallel to the Y axis. The segment S1 can be a third of the length of the sum of segment lengths, S1+S2, and the segment S2 can be two-thirds of the length of the sum S1+S2. In FIG. 2C, a second portion P2 can also be divided into two segments, S3 and S4, where S3 is parallel to the X axis and S4 is perpendicular to the X axis. The segment S3 can be a third of the length of the sum of segment lengths, S3+S4, and the segment S4 can be two-thirds of the length of the sum S3+S4. Generally, a second portion P2 can be two thirds of the total length of all portions of the conductive structure 210B, where P1 is one third of the total length (e.g. S1+S2=P1; S3+S4=P2; P2=2*P1). The angle θ can be specified to be 45 degrees as in the example of the conductive structure 210A of FIG. 2A. FIG. 2D illustrates a further permutation of the configuration of FIG. 2C, where an implantable medical device, implant 202D shown, can include a conductive structure 210D having first and second portions P1 and P2, similar to FIG. 2C in other respects, but where S3 is perpendicular to the X axis and S4 is parallel to the X axis. In other respects, the conductive structure 210D of FIG. 2D is similar to the example of FIG. 2C, such as located on or within a dielectric portion 208, located at an end of a housing 206, with segment S1 located proximal to a feed location 228.

Generally, use of respective portions comprising one third and two thirds of a total length (or phrased alternately as two portions having a two-to-one length ratio with respect to each other) provides a more uniform radiation pattern, in view of the non-uniform current density established in an antenna conductive structure. The shorter first portion located proximally to the feed location generally exhibits a higher current density during operation, relative to the second portion.

FIG. 3A and FIG. 3B illustrate further respective examples comprising implantable antenna assembly configurations, such as can be included in an implantable medical device or form a portion of an implantable medical device. In FIG. 3A, an implantable medical device, implant 302A shown, can include a dielectric portion 308 housing a conductive structure 310A, such as located on or embedded just below a face 330 of the dielectric portion 308. As in other examples, an angle θ can be specified to be 45 degrees to provide uniform radiation in at least two different polarization axes. Also, as in other examples, the conductive structure 310A can form a monopole configuration with a conductive portion of a housing 306 serving as a return conductor or counterpoise. Implant 302A can comprise feed location 328 which can be of similar construction and arrangement as feed location 228 described herein. Feed location 328 can include an insulated feedthrough structure, such as a coaxial feedthrough configuration providing a pathway for a signal to be fed to the conductive structure 310A from a transceiver located within a sealed (e.g. hermetically sealed) compartment within the housing 306.

In FIG. 3B, an implantable medical device, implant 302B shown, can include a dielectric portion 308 housing a conductive structure 310B, such as located on or embedded just below a face 330 of the dielectric portion 308. As in other examples, an angle θ can be specified to be 45 degrees to provide uniform radiation in at least two different polarization axes. Also, as in other examples, the conductive structure 310B can form a monopole configuration with a conductive portion of a housing 306 serving as a return conductor or counterpoise. By contrast with the example of the conductive structure 310A of FIG. 3A, the conductive structure 310B of FIG. 3B can include multiple segments, such as extending in various directions in a plane parallel (or coincident) with a face 330 of the dielectric portion 308, or extending in multiple directions (e.g. having segments extending along the X-Z plane parallel to the face 330, the Y-Z plane in the depth direction into the dielectric portion 308, or in other directions). In the examples of FIG. 3A and FIG. 3B, the dielectric portion 308 can be thinner than the illustrative examples of FIG. 2A through FIG. 2D, such as having a thinner profile in the Y dimension as shown in the X-Y-Z coordinate axis of FIG. 3A and FIG. 3B. Implant 302B can comprise feed location 328 which can be of similar construction and arrangement as feed location 228 described herein. Feed location 328 can include an insulated feedthrough structure, such as a coaxial feedthrough configuration providing a pathway for a signal to be fed to the conductive structure 310B from a transceiver located within a sealed (e.g. hermetically sealed) compartment within the housing 306.

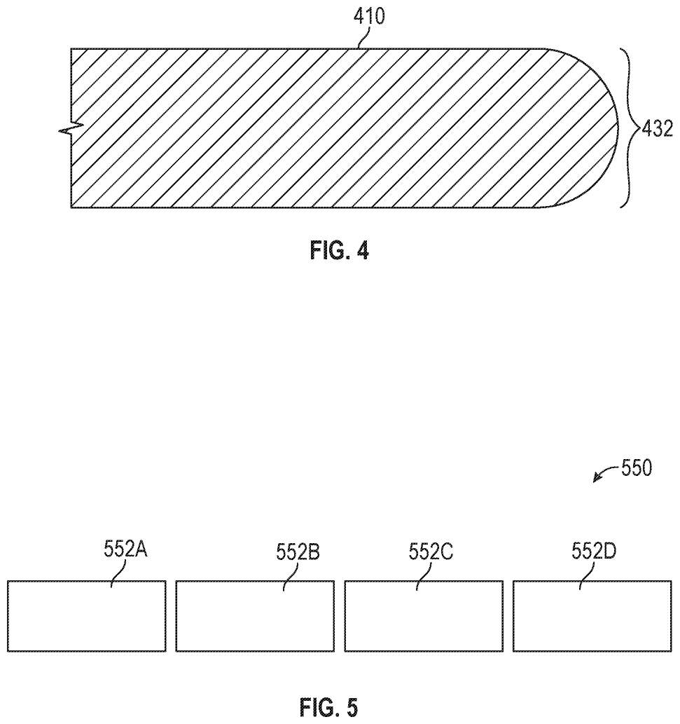

FIG. 4 illustrates generally an illustrative example of a distal end 432 (e.g. distal relative to a feed location) of a conductive structure 410 that can form a portion of an implantable antenna assembly. The distal end can be curved, radiused, and/or otherwise smoothed, such as to avoid sharp edges. Avoidance of sharp edges can help to reduce a likelihood of “hot spots” or high potential gradients when the conductive structure 410 is exposed to an externally-applied field, such as associated with magnetic resonance imaging (MRI) and/or another source of radio frequency and/or microwave energy. Other sources can include radar equipment, cellular base-station equipment, or broadcast transmitters, as illustrative examples. Use of a smoothed (e.g. radiused) end may also help provide compliance with Specific Absorption Ratio (SAR) limitations, such as when the conductive structure 410 forms part of a transmitting antenna structure. Generally, the conductive structure 410 can form a portion of an antenna assembly of the present inventive concepts, such as is shown and described elsewhere herein.

FIG. 5 illustrates generally an example comprising a sensor electrode structure, electrode structure 550 shown. As mentioned elsewhere herein, the implantable antenna assemblies of other examples can be included in an implantable medical device, such as a device having monitoring capabilities and/or therapeutic capabilities. For example, for electrocardiogram acquisition, electrode structures can be included as a portion of the implantable medical device. The present inventors have recognized, among other things, that use of a single, monolithic electrode structure may facilitate unwanted coupling and/or loading of a nearby antenna conductive structure. The electrode structure 550 of FIG. 5 can help suppress or inhibit such loading or coupling, such as by using a segmented configuration where the electrode structure 550 is divided into multiple conductive regions, such as conductive regions 552A, 552B, 552C, and 552D shown. The multiple conductive regions can capacitively couple with each other, but are otherwise conductively isolated from each other. Such an approach can provide an electrode configuration that still functions as a monolithic electrode for purposes of sensing physiologic signals (e.g. low frequency alternating current potentials of tens, hundreds, or thousands of Hertz), while suppressing resonant or loading effects in a frequency range used for wireless communication by a nearby antenna. Simulation results below illustrate how the segmented configuration or other factors such as antenna orientation relative to electrode structure 550 orientation, can affect loading or coupling.

FIG. 6A shows an illustrative example of a simulated surface current density for a dipole antenna configuration, antenna configuration 610 shown, at an operating frequency of 2.45 GHz. Generally, the highest current density is at the central region of the antenna configuration 610, near the feed point.

FIG. 6B shows an illustrative example of a simulated surface current density for a dipole antenna configuration, antenna configuration 610 shown, at an operating frequency of 2.45 GHz and an induced surface current density for a nearby unsegmented sensor electrode structure, electrode structure 651A shown. As shown in FIG. 6B, a current is induced in the electrode structure 651A and a location of a highest current density corresponds to the central region of the antenna configuration 610, with coupling maximized in view of the longitudinal orientation of the electrode structure 651A in parallel with the antenna configuration 610. By contrast, FIG. 6C shows an illustrative example of a simulated surface current density for a dipole antenna configuration, antenna configuration 610 shown, at an operating frequency of 2.45 GHz and a nearby segmented sensor electrode structure, electrode structure 650 shown. Segmentation suppresses or inhibits development of the induced current in the electrode structure 650. Alternatively, or in addition, as shown in FIG. 6D, a sensor electrode structure, electrode structure 651B shown, can be oriented in an orthogonal manner relative to at least a portion of an antenna structure (e.g. perpendicular to a long axis of a dipole antenna configuration, antenna configuration 610 as shown in FIG. 6D). The electrode structure 651B is not shown as segmented, but segmentation can also be combined with the perpendicular orientation to further reduce an induced current distribution from operation of antenna configuration 610.

FIG. 7A illustrates generally an example comprising a sensor electrode structure, electrode structure 750 shown, having two layers of conductive regions comprising 752A through 752N. The electrode structure 750 can include a feed and/or pickup node, node 770 shown, and signals can be coupled between adjacent conductive regions using capacitive coupling. As an illustrative example, two or more separate electrode structures (such as each having a configuration similar to the electrode structure 750) can be included in an implantable medical device, such as to provide electrodes for sensing of a physiologic signal such as potentials associated with cardiac activity. Alternatively, or in addition, a conductive housing can be used as an electrode (e.g. a “can” connection).

FIG. 7B shows an illustrative example of a simulated surface current density for a dipole antenna configuration, antenna configuration 710 shown, at an operating frequency of 2.45 GHz, fed at a feed location 728. FIG. 7B also shows a nearby two-layer segmented sensor electrode structure, electrode structure 750 shown. As in other examples described above, segmentation suppresses or inhibits development of the induced current in the electrode structure 750, and such suppression is also applicable to multi-layer structures such as the two-layer structure shown in FIG. 7B. Further variations are possible. For example, FIG. 8A, FIG. 8B, and FIG. 8C show respective illustrative examples of arrays of sensor electrode structures, electrode structures 850A, 850B, and 850C shown, having conductive regions extending in two directions (e.g. to provide a two-dimensional array structure). In the examples of FIG. 8A, FIG. 8B, and FIG. 8C, the conductive regions are shown as each being the same for each respective array, but this is not required. Combinations of different shapes can be used, and such shapes need not be planar (e.g. the electrode configurations can be arranged to conform to a curved surface), and/or such structures can include multiple layers in a manner similar to the example of FIG. 7B. Generally, the sensor electrode configurations shown herein can coexist with various antenna configurations, such as shown elsewhere herein.

The above detailed description includes references to the accompanying drawings, which form a part of the detailed description. The drawings show, by way of illustration, specific embodiments in which the invention can be practiced. These embodiments are also referred to generally as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. An implantable antenna assembly, comprising:
a feed location;
a dielectric portion; and
a conductive structure located on or within the dielectric portion, the conductive structure coupled to the feed location, the conductive structure comprising:
a first portion extending along a first plane; and
a second portion extending along a second plane, the second plane orthogonal to the first plane;
wherein at least one of the first or second portions projects in both a parallel and a perpendicular in-plane direction, with respect to an in-plane reference axis;
wherein the first portion comprises about one third of a total length including the first and second portions; and
wherein the second portion comprises about two thirds of the total length including the first and second portions.

2.-37. (canceled)
